(19) United States
(12) Patent Application Publication
(10) Pub. No.: US 2008/0154115 A1
(43) Pub. Date: Jun. 26, 2008

(54) Magnetic Resonance Imaging Method

(75) Inventors: Miha Fuderer, Eindhoven (NL); Holger Eggers, Kaltenkirchen (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 10/597,762

(22) PCT Filed: Feb. 3, 2005
(86) PCT No.: PCT/IB05/50458

§ 371 Date: Aug. 7, 2006

(30) Foreign Application Priority Data
Feb. 10, 2004 (EP) 04100486.2

(51) Int. Cl. A61B 5/055

(52) U.S. Cl. 600/410; 324/309

(57) ABSTRACT

A novel magnetic resonance imaging method and apparatus is described wherein an image is derived from sub-sampled magnetic resonance signals and on the basis of the spatial sensitivity profile of each receiving antenna. A sequence of RF-pulses and gradients is applied, which sequence corresponds to a set of trajectories containing at least one substantially non-linear trajectory in k-space, wherein the density of said trajectory set being substantially lower than the density corresponding to the object size. Each signal along said trajectory set is sampled at least at two different receiver antenna positions. The image is reconstructed by converting the data of said signals to a Cartesian grid by convolution with a gridding kernel, whereby the gridding kernel is specific for each antenna, differs between one region and another in k-space, and is a Fourier-transform of a pattern weighted for each antenna with respect to the Cartesian grid.

The invention relates to a magnetic resonance (MR) method for forming an image of an object wherein a set of non-linear trajectories in k-space is acquired, whereas the density of said set of trajectories is substantially lower than the density corresponding to the object size. Signals along these trajectories are sampled by means of one or more receiving antennae, and a magnetic resonance image is derived from these signals and on the basis of the spatial sensitivity profile of the set of receiving antennae. The invention notably pertains to a magnetic resonance imaging method in which magnetic resonance signals are acquired by means of a receiver antennae system and a magnetic resonance image is reconstructed on the basis of the magnetic resonance signals.

Such a magnetic resonance imaging method is known from the international application WO 01/73463.

In this known magnetic resonance imaging method the magnetic resonance signals are acquired by scanning along a trajectory in k-space. The known magnetic resonance imaging method offers a high degree of freedom in choosing the acquisition trajectory to be followed through k-space. Notably, acquisition trajectories, notably spiral shaped trajectories, which give rise to irregular sampling patterns in k-space may be used.

The invention also relates to an MR apparatus and a computer program product for carrying out such a method.

Normally, in parallel imaging as SENSE (Pruessmann) or SMASH (Sodickson) the reconstruction of the image is performed by a Cartesian gridding of k-space or image space, respectively.

In US-A-2003/0122545 a magnetic resonance imaging method is described wherein the degree of sub-sampling is chosen such that the ensuing acquisition time for receiving (echo) series of magnetic resonance signals due to an individual RF excitation is shorter than the decay time of the MR signals. Preferably, a segmented scan of the k-space is performed, the number of segments and the number of lines scanned in each segment being adjustable and a predetermined total number of lines being scanned. A small number of segments is used such that the acquisition time for receiving the magnetic resonance signals for the complete magnetic resonance image is shorter than the process time of the dynamic process involved. Although the method is described with a scanning trajectory of straight lines in k-space also other trajectories like curved lines as arcs of circle or spirals could be possible. However, in such case more complex frequency and phase encoding of the magnetic resonance signals will be required. A specific solution for continuous non-Cartesian trajectories in k-space is not described.

In an article of M. Bydder et. al. in Magn. Reson. Med 10 (2002) it is mentioned that partially parallel imaging techniques for reconstruction of under-sampled k-space data from multiple coils may be used with arbitrary acquisition schemes (e.g. Cartesian, spiral etc.) by casting the problem as a large linear system of equations. For realistic applications, however, the computational costs for solving this system directly is prohibitive. To date there is no realistic solution for a procedure of fast reconstruction of grossly undersampled data on a continuous non-Cartesian trajectory especially as spiral sampling in k-space.

An object of the present invention is to further reduce the computational effort involved in the reconstruction of the magnetic resonance imaging method from the acquired magnetic resonance signals.

This object is achieved by the magnetic resonance imaging method of the invention, wherein
magnetic resonance signals are acquired by means of a receiver antennae system via a plurality of signal channels
   which receiver antennae system has a sensitivity profile
the magnetic resonance signals are acquired with undersampling
   for respective orientated sector shaped regions in k-space, regularly re-sampled magnetic resonance signals are re-sampled on a regular sampling grid from the undersampled acquired magnetic resonance signals
   the re-sampling involving convolution of the undersampled acquired magnetic resonance signals by a gridding kernel
      the gridding kernel depending on
      the orientation of the sector shaped region at issue and
      the sensitivity profile of the receiver antennae system and
a magnetic resonance image is reconstructed from the re-sampled magnetic resonance signals.

The present invention is based on the following insights. In order to achieve a fast reconstruction, e.g. techniques such as fast Fourier transformation (FFT) are employed. As input, these techniques require that data are sampled on a regular sampling grid. Further, a wide class of acquisition trajectories in k-space, notably spiral shaped trajectories and trajectories that include spiral segments are accurately or at least fairly approximated by (almost) parallel segments of the trajectory in respective sector shaped regions of k-space. In general, sector shaped regions may be regions of k-space which have a main axis that passes through the origin of k-space. Such sector shaped regions extend between angular boundaries, that is between a respective minimum and maximum modulus of the k-vector to the periphery of k-space and are bounded by radial boundaries that extend radially from the origin of k-space. The sector shaped regions maybe full sectors which extend from or through the origin of k-space into the periphery of k-space. In two dimensions the sector shaped regions are flat sectors or sector segments or sectors that extend point-symmetrically through the origin of k-space, in three dimensions the sector shaped regions are cones or portions of cones in k-space. According to the invention, the reconstruction which involves a re-gridding to re-sample the acquired magnetic resonance signals to re-sampled magnetic resonance signals on the regular grid is performed separately for the individual sector shaped regions. The re-gridding involves a convolution with a gridding kernel. The gridding kernel depends on the orientation of the sector shaped region at issue so as to account for the appropriate direction in the image space into which aliasing will occur due to the Fourier relationship between pixel-values of the magnetic resonance image and the re-sampled magnetic resonance signals in k-space. Further, the gridding kernel involves the sensitivity profile of the receiver antennae system in order to take account of aliasing that is caused by undersampling of the acquired magnetic resonance signals. To derive the gridding kernel on the basis of the orientation of the sector shaped region and on the basis of the sensitivity profile does not require much computational effort. The computational effort is notably reduced because aliasing is caused by a comparatively small number of pixels or voxels. Accordingly, matrix inversions are only needed for matrices having a relatively low dimensionality. The actual re-sampling onto the regular grid, such as a Cartesian square lattice, involves only convolution with the gridding kernel which takes only little computational effort. The final reconstruction of the magnetic resonance image is then performed by a FFT technique that takes only a short computation time.

It appears that the dependence on the orientation of the sector shaped region in k-space of the gridding kernel involves a quite smooth variation. Accordingly, the gridding kernel for a particular orientation is also accurately valid for rather wide sectors in k-space.

It is also an object of the present invention to provide a magnetic resonance imaging method enabling a fast reconstruction of grossly undersampled non-Cartesian sampling in k-space, especially along a spiral trajectory. It is a further object of the present invention to provide a system and a computer program product for performing such a method.

This object is achieved by means of a magnetic resonance imaging method according to the invention as claimed in particular in claims 1, 2 and 3.

These objects are achieved by a method as claimed in claim 1, by an MR apparatus as claimed in claim 6 and by a computer program product as claimed in claim 21.

It is a main advantage of the present invention that formulations of SPACE-RIP and non-Cartesian SENSE are derived that represent coil sensitivity information in the Fourier domain. Due to the small number of Fourier terms required, the linear system is highly sparse and so allows efficient solution of the equations. Thus, spiral scanning is made feasible at a high degree of undersampling so that a very fast acquisition and reconstruction is achieved.

The main aspect of the present invention is that a non-Cartesian trajectory in k-space can be described locally by a coordinate system of imaginary parallel tangential lines which form locally a Cartesian grid in order to perform subsampling like SENSE or SMASH. If the whole k-space is subdivided by rays divided homogeneously over an angle of 360° a continuous system of local Cartesian grids is obtained. These parts of k-space are than locally reconstructed and converted as a whole to an image.

This and other advantages of the invention are disclosed in the dependent claims and in the following description in which an exemplified embodiment of the invention is described with respect to the accompanying drawings. Therein shows:

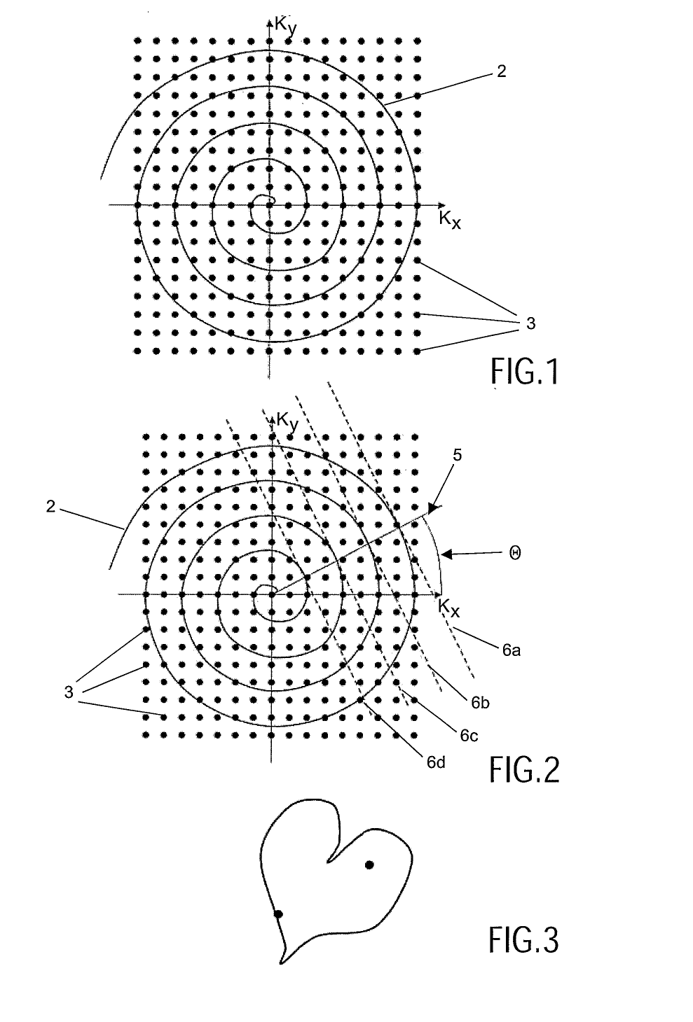

FIG. 1 an undersampled spiral trajectory in k-space,

FIG. 2 the same spiral trajectory as in FIG. 1 with hypothetical parallel scan lines for a region around the radius with an angle θ,

FIG. 3 folding points in the image corresponding to a hypothetical Cartesian sampling pattern as shown in FIG. 2,

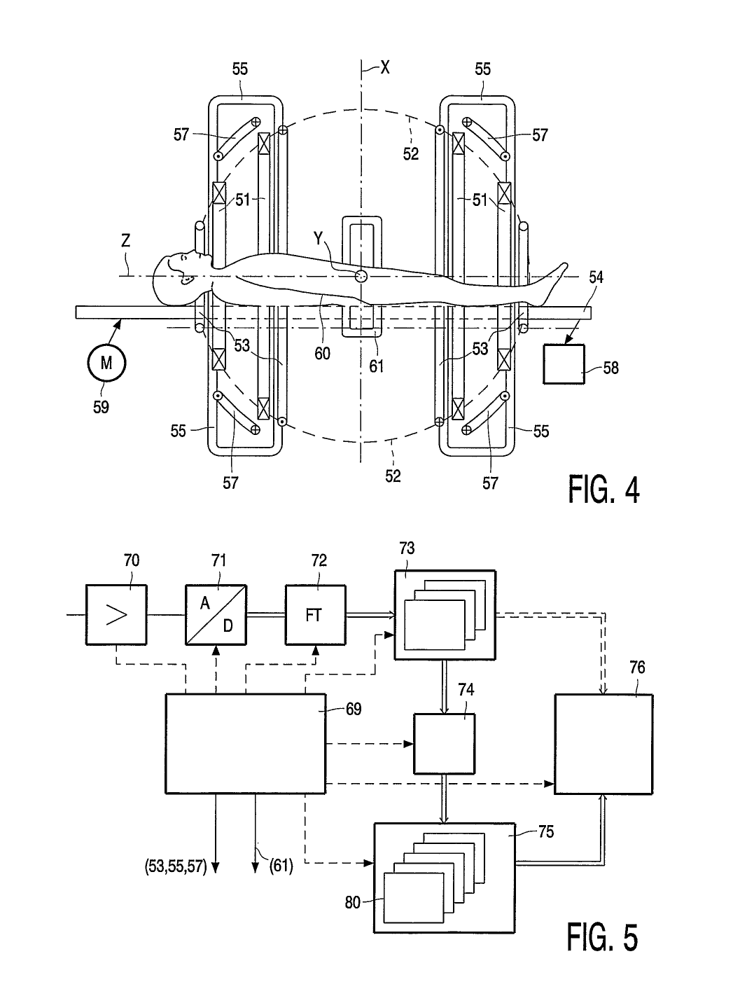

FIG. 4 an apparatus for carrying out the method in accordance with the present invention, and

FIG. 5 a circuit diagram of the apparatus as shown in FIG. 4.

### BASIS OF THE PRESENT INVENTION

In FIG. 1 a spiral scan trajectory has been depicted, which is a single spiral arm 2 in single shot EPI. The dots 3 represent a Cartesian grid of a density that would be required to properly image the Field-of-View (FOV) encompassing the object to be imaged. The actual density may also be slightly higher (so called “overgridding”), corresponding to a region that is slightly larger than the object. The spiral arm 2 has been grossly undersampled according to the SENSE method with an undersampling factor of about two. This can immediately be seen from FIG. 1 as the distance between the spiral parts of the arm is at about a distance of two dots 3. From the point of view of the Nyquist criterion this is insufficient sampling. However, if the signal of that trajectory has been sampled by at least two receiver antennae or coils having different spatial-sensitivity patterns, the image can be reconstructed nevertheless. The reconstruction of the image requires the solution of a set of approximately N equations with N unknowns, where N is of the order of magnitude of the number of sample points times the number of coils, or of the order of the number of pixels in the resulting image (i.e., N is about 10⁴ to 10⁵). This means that a number of N equations should be solved, which is not feasible by direct matrix inversion. Therefore, an iterative solution is proposed by several authors, which requires about ten iterations, each involving expensive computational gridding operations.

### Outline of the Algorithm According to the Present Invention

In FIG. 2 in the spiral arm 2 a radial line 5 with an angle θ is depicted, which traverses the spiral arm 2. At the crossing points between the radial line 5 and the spiral arm 2 tangential lines 6a, 6b, 6c and 6d are drawn, which show that the neighboring parts of spiral arm 2 are more or less parallel and equidistant. At present an Archimedic spiral is shown. but also other spiral functions may be used. This situation is well known form the Cartesian approach in parallel imaging: if these equidistant lines 6a to 6b would have covered the whole k-space, then reconstruction would be much less laborious. In image space a discrete number of object-points would “simply” be folded onto each other, as shown just for simplicity with two points in FIG. 3.

In principle, the problem can be solved then by “normal” SENSE reconstruction. This can be written as sum of receiver antenna signals mₖ (X, Y) “weighted” to a function aₖ (X, Y). This can also be written in the Fourier-domain as

$$\begin{matrix}p ( X , Y ) = {\sum }_{coils k} {F}^{- 1} \{ {α}_{k} ( {k}_{x} , {k}_{y} ) ⊗ {μ}_{k} ( {k}_{x} , {k}_{y} ) \} , or & ( 1 ) \\ p ( X , Y ) = {F}^{- 1} \{ {\sum }_{coils k} {α}_{k} ( {k}_{x} , {k}_{y} ) ⊗ {μ}_{k} ( {k}_{x} , {k}_{y} ) \} & ( 2 )\end{matrix}$$

with μₖ (kₓ, k_{y}) the measured data along the hypothetical equidistant lines 6a to 6b, with α the Fourier transform of aₖ (X, Y).

It is noted that equations (1) or (2) describe exactly the same operations performed for normal spiral imaging without undersampling (SENSE, SMASH). There, the meaning of aₖ (X, Y) is the “gridding kernel”, which is in essence the Fourier transform of a box (but tapered with smooth edges to prevent that aₖ (X, Y) having a large support).

In the present case, the shape of aₖ (X, Y) is not a tapered box, but a “reconstructing function”, which depends essentially on the coil sensitivity pattern of all receiver antennae or coils, on the folding distance of the SENSE method and eventually partly on the object shape (due to regularization). Yet, since the coil sensitivity functions are expected to be smooth functions in space, the functions aₖ (X, Y) are also expected to be smooth in space. For that reason, the gridding function αₖ (kₓ, k_{y}) is expected to have a relatively small support. It is supposed that a support of 12*12 to 16*16 Cartesian points will be sufficient (where for gridding of normal imaging a support of 4*4 is usually enough).

The obtained gridding function αₖ (kₓ, k_{y}) can be applied perfectly to reconstruct data from a set of parallel equidistant lines that are angulated with respect to the required grid. However, in this case the data is sampled along a spiral arm, and not along a line. That means that the obtained gridding kernel is only valid for points that are strictly positioned on the radius with an angle θ. Strictly the gridding kernel aₖ (X, Y) should be calculated for an infinity of situations. Yet, coil sensitivity patterns are normally smooth functions of space. This means that the weighting function aₖ (X, Y) (and consequently the gridding function αₖ (kₓ, k_{y})) will not alter significantly if the folding direction is slightly changed. The “folding direction” is defined by the angle between the line of the folding points, or, equivalently, by the orientation of the hypothetical parallel lines 6a to 6d. For that reason, the obtained gridding function can be applied in a predetermined region around the radius with angle θ. This allows to calculate αₖ (kₓ, k_{y}) for a limited number of radii (e.g. 10 or 20).

### Resulting Algorithm

It is assumed that coil sensitivities are known in the entire relevant region, and that there is some knowledge on the presence of the object (as in Cartesian SENSE). Given is a spiral trajectory in two-dimensional k-space. The only relevant issue is then the distance between the spiral arms. Reconstruction according to the present invention will be performed by following steps:
1. A Cartesian grid is chosen. The distance (or density) should correspond to the size of the object under study.
2. A number of equidistant radii over the k-space is defined (e.g. 10 or 20).
3. For each radius, the direction of the lines 6a to 6b tangential to the spiral arms is determined, whereas the distance between the tangential lines should be independent of the angle.
4. For the obtained set of folding points, the SENSE reconstruction matrix A=(S^{h}·Ψ⁻¹S+R⁻¹)⁻¹·S^{h}·Ψ⁻¹ is calculated, wherein S is the receiver antenna or coil sensitivity matrix, Ψ is the noise covariance between the coil channels, R is the regularization matrix and S^{h} means the hermitian conjugate of S. The segments of the matrix A are combined into the function aₖ (X, Y) for each receiver antenna or coil k. (It is noted that this step is part of the normal SENSE reconstruction.)
5. Outside of the image area, the function aₖ (X, Y) is set to zero (zero-padding); a Fourier transformation of this function is then performed into αₖ (kₓ, k_{y}), or any other suitable method to interpolate values of αₖ (kₓ, k_{y}) on a sufficiently fine grid for the subsequent convolution and resampling is used. This function is expected to be relevant only for small values of kₓ and k_{y}, so that the outer parts thereof can be discarded.
6. The data is acquired.
7. The sampling density compensation is performed.
8. For each point along the spiral trajectory, the radius that comes closest to that sample point is determined
9. Using the gridding function αₖ (kₓ, k_{y}) calculated for the closest radius of each coil, and the acquired data sample of each coil, a gridding operation is performed. This operation is part of the normal spiral scan reconstruction procedure, only the extent of αₖ may be larger.
10. The data is summed over the coil elements.
11. The Cartesian grid points are Fourier-transformed.

It is noted that for dynamic scans (or any type of scans in which a multitude of data sets for the same geometric positions is acquired), steps 1 to 5 have to done only once.

### Refinements of the Algorithm

R1. A grid can be chosen which does not correspond to the normal FOV (or “the size of the object under study”) but to a slightly larger FOV, and thus being slightly denser. The margins between the user selected FOV and the larger or “overgridded” FOV are known to contain no object. Towards the edge of the overgridded FOV, the regularization values R are gradually forced to zero. In such a manner, discontinuities of the functions aₖ (X, Y) are avoided, leading to a smaller support of αₖ (kₓ, k_{y}).
R2. The functions aₖ (X, Y) can be preconditioned by first multiplying them with a common shaping function, in order to reduce the support of αₖ (kₓ, k_{y}). This may be a tapering window function, or a multiplication by e.g. the sum of squares of sensitivities, to prevent huge values on points in space where all coils are insensitive.
R3. The two nearest radii can be taken and the gridding kernel functions between them can be interpolated. In a more efficient way, both radii are gridded and the result thereof is interpolated.
R4. The most central region of k-space can be reconstructed by another method (e.g. direct inversion) and the gridding result is blended with the alternative reconstruction of the central region.
R5. The functions αₖ (X, Y) are divided into a defined number of subfunctions, for which the support of the corresponding \tilde{α}ₖ (kₓ, k_{y}) is particularly small, and the sum of which equals or approximates the original aₖ (X, Y) in the full Field-of-View. This allows to cope with sharp transitions in aₖ (X, Y), which most notably occur at the edges of the reduced Field-of-Views. A separate gridding for each set of subfunctions is required in this case. Compared with expanding the size of the local convolution kernel adequately to represent sharp transitions, this may be computationally attractive. One natural choice would be to take each reduced Field-of-View separately, and treat the function—with or without periodic replication—with some sort of tapering window function.
R6. For a similar reason, sets of samples assigned to adjacent radii can be separately gridded and transformed. This allows to provide “space” for errors with the full FOV, very much like the conventional oversampling provides “space” for errors at the edges of the full FOV.
R7. The proposed algorithm may be used to generate an image with which one of the known iterative reconstruction algorithms (e.g. the conjugate gradient method) is then initialized. This allows to substantially reduce the number of iterations required by these methods to achieve an adequate image quality. Equally, this strategy permits to eliminate any artifacts potentially remaining with the proposed algorithm for reasonable parameter settings (i.e. limited support of αₖ (kₓ, k_{y}) and limited number of radii).

### Extension of the Method

In principle, the local neighborhood of each k-space sampling point and the local degree of subsampling may be considered separately. In this case, steps 1 to 5 of the method according to the present invention would be performed for sets of points with similar local properties, which may be arbitrarily distributed in k-space. This would allow to apply the proposed algorithm also to, among others, variable density spiral and conventional radial acquisitions.

The apparatus shown in FIG. 4 is an MR apparatus which comprises a system of four coils 51 for generating a steady, uniform magnetic field whose strength is of the order of magnitude of from some tenths of Tesla to some Tesla. The coils 51, being concentrically arranged relative to the z axis, may be provided on a spherical surface 52. The patient 60 to be examined is arranged on a table 54 which is positioned inside these coils. In order to produce a magnetic field which extends in the z direction and linearly varies in this direction (which field is also referred to hereinafter as the gradient field), four coils 53 as multiple receiver antennae are provided on the spherical surface 52. Also present are four coils 57 which generate a gradient field which also extends (vertically) in the x direction. A magnetic gradient field extending in the z direction and having a gradient in the y direction (perpendicularly to the plane of the drawing) is generated by four coils 55 which may be identical to the coils 57 but are arranged so as to be offset 900 in space with respect thereto. Only two of these four coils are shown here.

Because each of the three coil systems 53, 55, and 57 for generating the magnetic gradient fields is symmetrically arranged relative to the spherical surface, the field strength at the center of the sphere is determined exclusively by the steady, uniform magnetic field of the coil 51. Also provided is an RF coil 61 which generates an essentially uniform RF magnetic field which extends perpendicularly to the direction of the steady, uniform magnetic field (i.e. perpendicularly to the z direction). The RF coil receives an RF modulated current from an RF generator during each RF pulse The RF coil 61 can also be used for receiving the spin resonance signals generated in the examination zone.

As is shown in FIG. 5 the MR signals received in the MR apparatus are amplified by a unit 70 and transposed in the baseband. The analog signal thus obtained is converted into a sequence of digital values by an analog-to-digital converter 71. The analog-to-digital converter 71 is controlled by a control unit 69 so that it generates digital data words only during the read-out phase. The analog-to-digital converter 71 is succeeded by a Fourier transformation unit 72 which performs a one-dimensional Fourier transformation over the sequence of sampling values obtained by digitization of an MR signal, execution being so fast that the Fourier transformation is terminated before the next MR signal is received.

The raw data thus produced by Fourier transformation is written into a memory 73 whose storage capacity suffices for the storage of several sets of raw data. From these sets of raw data a composition unit 74 generates a composite image in the described manner; this composite image is stored in a memory 75 whose storage capacity suffices for the storage of a large number of successive composite images 80. These sets of data are calculated for different instants, the spacing of which is preferably small in comparison with the measurement period required for the acquisition of a set of data. A reconstruction unit 76, performing a composition of the successive images, produces MR images from the sets of data thus acquired, said MR images being stored. The MR images represent the examination zone at the predetermined instants. The series of the MR images thus obtained from the data suitably reproduces the dynamic processes in the examination zone.

The units 70 to 76 are controlled by the control unit 69. As denoted by the down wards pointing arrows, the control unit also imposes the variation in time of the currents in the gradient coil systems 53, 55 and 57 as well as the central frequency, the bandwidth and the envelope of the RF pulses generated by the RF coil 61. The memories 73 and 75 as well as the MR image memory (not shown) in the reconstruction unit 76 can be realized by way of a single memory of adequate capacity. The Fourier transformation unit 72, the composition unit 74 and the reconstruction unit 76 can be realized by way of a data processor well-suited for running a computer program according the above mentioned method.

1. A magnetic resonance imaging method wherein
magnetic resonance signals are acquired by means of a receiver antennae system via a plurality of signal channels
which receiver antennae system has a sensitivity profile
the magnetic resonance signals are acquired with undersampling
for respective orientated sector shaped regions in k-space, regularly re-sampled magnetic resonance signals are re-sampled on a regular sampling grid from the undersampled acquired magnetic resonance signals
the re-sampling involving convolution of the undersampled acquired magnetic resonance signals by a gridding kernel
the gridding kernel depending on
the orientation of the sector shaped region at issue and
the sensitivity profile of the receiver antennae system and
a magnetic resonance image is reconstructed from the re-sampled magnetic resonance signals.

2. A magnetic resonance imaging method as claimed in claim 1, wherein the magnetic resonance signals are acquired by scanning k-space along a non-linear, in particular spiral shaped, trajectory.

3. A magnetic resonance imaging method as claimed in claim 1 for forming an image of an object wherein
a magnetic resonance image is derived from sub-sampled magnetic resonance signals and on the basis of the spatial sensitivity profiles of a plurality of receiving antennae,
a sequence of RF-pulses and gradients is applied, which sequence corresponds to a set of trajectories comprising at least one substantially non-linear trajectory in k-space, wherein the sampling density of said trajectory set being substantially lower than the normal sampling density corresponding to the object size,
each signal along said trajectory set is sampled at least at two different receiver antenna positions, resulting into a plurality of receiver-antenna signals,
the image is reconstructed by converting the data of said signals from said trajectory set to a Cartesian grid by convolution with a gridding kernel,
and whereby
the gridding kernel is a Fourier-transform of a pattern weighted for each antenna with respect to the Cartesian grid, and
the gridding kernel pattern differs between one region and another in k-space.

4. A method as claimed in claim 1, wherein the weighting pattern is obtained in that
to every individual region of k-space, a set of parallel equidistant lines is assigned, which lines locally match said trajectory set,
a pattern of overlapping points in image space is determined, which corresponds to the set of parallel equidistant lines in k-space,
in image space, the weighting pattern per antenna is calculated, which pattern approximately corresponds to a pattern solely of said parallel equidistant lines in the individual region of k-space.

5. A method as claimed in claim 2, wherein at least part of the trajectory set corresponds to an Archimedic spiral and the regions in k-space are defined by their azimuthal angle in k-space.

6. A method as claimed in claim 4, wherein the weighting pattern of the antenna is calculated according to the inversion of a Cartesian set of equations for the subsampled data and the spatial sensitivity profiles of the receiving antennae.

7. A method as claimed in claim 6, wherein the inversion of said Cartesian set of equations is formulated as A=(S^{h}·Ψ⁻¹·S+R⁻¹)⁻¹·S^{h}·Ψ⁻¹, wherein A is the reconstruction matrix, S is the receiver antenna sensitivity matrix (s_{i j}), wherein s_{i j} is the spatial sensitivity profile of antenna i on the j-th point of the overlapping set of points, Ψ is the noise covariance between the antennae, R is the regularization matrix and S^{h} means the hermitian conjugate of S.

8. A method as claimed in claim 7, wherein the gridding kernel is chosen to correspond to a larger FOV as the normal FOV covering the size of the object to be studied and the values of the regularization matrix R between the margin of the larger FOV and the normal FOV are set to zero.

9. A method as claimed in claim 8, wherein the gridding kernel pattern for each antenna derived from the reconstruction matrix A is multiplied with a common shaping function comprising a tapering window function or the sum of squares of sensitivities of each antenna.

10. A method as claimed in claim 5, wherein the gridding kernel functions between the two nearest radii traversing the spiral trajectory set are interpolated.

11. A method as claimed in claim 10, wherein both radii are gridded and the result thereof is interpolated.

12. A method as claimed in one of claim 1, wherein the most central region of k-space is reconstructed at full sampling density by direct inversion and the result of the gridding reconstruction method is blended with the result of the reconstruction at full sampling density.

13. A method as claimed in claim 7, wherein the gridding kernel pattern for each antenna derived from the reconstruction matrix A is divided into a defined number of subfunctions, for which the support of the corresponding functions in k-space tends to zero, in order to discard sharp transitions in the gridding kernel pattern, whereas each subfunction is gridded separately.

14. A method as claimed in claim 10, wherein sets of samples assigned to adjacent radii are gridded and transformed separately.

15. Use of the image generated by the method as claimed in claim 1, in order to initialize a conventional iterative algorithm for reconstruction of the image.

16. A magnetic resonance imaging apparatus for obtaining an MR image from a plurality of signals comprising:
a main magnet,
a transmitter antenna for excitation of spins in a predetermined area of the patients
a plurality of receiver antennae for sampling signals in a restricted homogeneity region of the main magnet field,
a table for bearing a patient,
means for continuously moving the table through the bore of the main magnetic,
means for deriving a magnetic resonance image from sub-sampled magnetic resonance signals and on the basis of the spatial sensitivity profile of each of said receiving antenna positions,
means for applying a sequence of RE-pulses and gradients, which sequence corresponds to a set of trajectories comprising at least one substantially non-linear trajectory in k-space, wherein the density of said trajectory set being substantially lower than the density corresponding to the object size,
means for sampling each signal along said trajectory set at least at two different receiver antenna positions, resulting into a plurality of receiver-antenna signals,
means for reconstructing the image by converting the data of said signals from said trajectory set to a Cartesian grid by convolution with a gridding kernel,
and whereby
the gridding kernel is specific for each antenna,
the gridding kernel pattern differs between one region and another in k-space, and
the gridding kernel is a Fourier-transform of a pattern weighted for each antenna with respect to the Cartesian grid.

17. Apparatus according to claim 16, further comprising
means for obtaining the weighting pattern including
means for assigning, to every individual region of k-space, a set of parallel equidistant lines, which lines locally match said trajectory set,
means for determining a pattern of overlapping points in image space, which corresponds to the set of parallel equidistant lines in k-space, and
means for calculating, in image space, the weighting pattern per antenna, which pattern approximately corresponds to a pattern solely of said parallel equidistant lines in the individual region of k-space.

18. Apparatus as claimed in claim 17, further comprising means for defining the regions in k-space by their azimuthal angle in k-space, whereas at least part of the trajectory set corresponds to equidistant spirals.

19. Apparatus method as claimed in claim 17, further comprising means for calculating the weighting pattern of the antenna according to the inversion of a Cartesian set of equations for the subsampled data and the spatial sensitivity profiles of the receiving antennae.

20. Apparatus as claimed in claim 19, whereas said means for calculating the inversion of said Cartesian set of equations is based on formula A=(S^{h}·Ψ⁻¹·S+R⁻¹)⁻¹·S^{h}·Ψ⁻¹, wherein A is the reconstruction matrix, S is the receiver antenna sensitivity matrix (s_{i j}) wherein s_{i j} is the spatial sensitivity profile of antenna i on the j-th point of the overlapping set of points, Ψ is the noise covariance between the antennae, R is the regularization matrix and S^{h} means the hermitian conjugate of S.

21. A computer program product stored on a computer usable medium for forming an image by means of the magnetic resonance method, comprising a computer readable program means for causing the computer to control the execution:
creating a main magnetic field by a main magnet,
excitation of spins in a predetermined area of the patient by a transmitter antenna,
sampling a plurality of signals in a restricted homogeneity region of the main magnet field at a plurality of receiver antenna positions,
continuously moving a table bearing a patient through the bore of the main magnet,
deriving a magnetic resonance image from sub-sampled magnetic resonance signals and on the basis of the spatial sensitivity profile of each of said receiving antenna positions,
applying, a sequence of RF-pulses and gradients, which sequence corresponds to a set of trajectories containing at least one substantially non-linear trajectory in k-space, wherein the density of said trajectory set being substantially lower than the density corresponding to the object size,
sampling each signal along said trajectory set at least at two different receiver antenna positions, resulting into a plurality of receiver-antenna signals,
reconstructing the image by converting the data of said signals from said trajectory set to a Cartesian grid by convolution with a gridding kernel,
and whereby
the gridding kernel is specific for each antenna,
the gridding kernel pattern differs between one region and another in k-space, and the gridding kernel is a Fourier-transform of a pattern weighted for each antenna with respect to the Cartesian grid.
